SEQUENCE LISTING (7 sequences)

SEQ ID NO: 1 - DNA, 1605 bp, Artificial Sequence
  made by chemical synthesis
  gene (1)..(1605): sequence encoding for CRM197, artificial gene optimised for expression in E. coli
ggtgccgatgacgtggttgactcttccaaaagcttcgtcatggaaaacttcagctcctatcacggcactaaaccgggttatgtcgacagcatccagaaaggcatccagaaaccgaaatctggcactcagggtaactatgacgacgactggaaagagttctactctaccgacaacaaatacgacgcggctggttattctgtggacaacgaaaacccgctgtctggtaaagctggtggtgttgttaaagtgacctacccgggtctgaccaaagttctggctctgaaagtggacaacgccgaaaccatcaaaaaagaactgggtctgtctctgaccgaaccgctgatggaacaggtaggtaccgaggaattcatcaaacgttttggtgatggtgcgtcccgtgttgtactgtctctgccatttgccgaaggttctagctctgtcgagtacatcaacaactgggagcaggccaaagctctgtctgtggaactggaaatcaacttcgagacccgtggtaaacgtggtcaggacgcaatgtatgaatacatggcacaggcttgcgcgggtaaccgtgtacgtcgttctgtaggttcttccctgtcttgcatcaacctggactgggatgtcatccgtgacaaaaccaaaaccaaaatcgagtccctgaaagagcacggtccgatcaaaaacaaaatgagcgaatctccgaacaaaacggtctctgaggaaaaagcgaaacagtacctggaagaattccatcagaccgccctggaacacccggaactgtctgaactgaaaaccgttaccggtactaacccggttttcgcaggtgctaactacgcagcgtgggcggttaacgtagcccaggtaatcgattccgaaaccgcagacaacctggaaaaaacgactgcggctctgtctattctgccgggtattggtagcgtgatgggtattgcagatggtgcagttcaccacaacacggaagaaatcgttgcgcagtctatcgctctgtcttctctgatggtagcacaggcgatcccgctggttggtgaactggttgacattggcttcgcggcctacaacttcgttgaatccatcatcaacctgttccaggttgtgcacaactcttacaaccgtccagcttactctccgggtcacaaaacccagccgttcctgcacgacggttatgcggtttcttggaacaccgttgaagacagcatcatccgtactggtttccagggtgaatctggccacgacatcaaaatcactgctgaaaacaccccgctgccgatcgcaggtgttctcctgccaactattccgggtaaactggacgtgaacaaatccaaaacgcacatctccgtgaacggtcgtaaaatccgcatgcgttgtcgtgcgattgatggtgacgttactttctgtcgtccgaaatctccggtctacgtaggtaacggtgtacatgctaacctccatgtagcgttccaccgttcttcttccgagaaaatccactccaacgagatctctagcgactctatcggtgttctgggttaccagaaaaccgttgaccacaccaaagtgaactccaaactcagcctgttcttcgaaatcaaatct

SEQ ID NO: 2 - DNA, 1689 bp, Artificial Sequence
  made by synthesis
  gene (1)..(87): encoding for HisTag
  gene (1)..(1689): encoding for CRM197-HisTag fusion protein
  misc_signal (73)..(87): encoding for DDDDK restriction site for enterokinase
  gene (88)..(1689): SEQ ID 1 encoding for CRM197 in E. coli
atgggtggttctcatcatcaccatcatcacggcatggcatctatgactggtggtcagcagatgggtcgtgatgacgatgacaaaggtgccgatgacgtggttgactcttccaaaagcttcgtcatggaaaacttcagctcctatcacggcactaaaccgggttatgtcgacagcatccagaaaggcatccagaaaccgaaatctggcactcagggtaactatgacgacgactggaaagagttctactctaccgacaacaaatacgacgcggctggttattctgtggacaacgaaaacccgctgtctggtaaagctggtggtgttgttaaagtgacctacccgggtctgaccaaagttctggctctgaaagtggacaacgccgaaaccatcaaaaaagaactgggtctgtctctgaccgaaccgctgatggaacaggtaggtaccgaggaattcatcaaacgttttggtgatggtgcgtcccgtgttgtactgtctctgccatttgccgaaggttctagctctgtcgagtacatcaacaactgggagcaggccaaagctctgtctgtggaactggaaatcaacttcgagacccgtggtaaacgtggtcaggacgcaatgtatgaatacatggcacaggcttgcgcgggtaaccgtgtacgtcgttctgtaggttcttccctgtcttgcatcaacctggactgggatgtcatccgtgacaaaaccaaaaccaaaatcgagtccctgaaagagcacggtccgatcaaaaacaaaatgagcgaatctccgaacaaaacggtctctgaggaaaaagcgaaacagtacctggaagaattccatcagaccgccctggaacacccggaactgtctgaactgaaaaccgttaccggtactaacccggttttcgcaggtgctaactacgcagcgtgggcggttaacgtagcccaggtaatcgattccgaaaccgcagacaacctggaaaaaacgactgcggctctgtctattctgccgggtattggtagcgtgatgggtattgcagatggtgcagttcaccacaacacggaagaaatcgttgcgcagtctatcgctctgtcttctctgatggtagcacaggcgatcccgctggttggtgaactggttgacattggcttcgcggcctacaacttcgttgaatccatcatcaacctgttccaggttgtgcacaactcttacaaccgtccagcttactctccgggtcacaaaacccagccgttcctgcacgacggttatgcggtttcttggaacaccgttgaagacagcatcatccgtactggtttccagggtgaatctggccacgacatcaaaatcactgctgaaaacaccccgctgccgatcgcaggtgttctcctgccaactattccgggtaaactggacgtgaacaaatccaaaacgcacatctccgtgaacggtcgtaaaatccgcatgcgttgtcgtgcgattgatggtgacgttactttctgtcgtccgaaatctccggtctacgtaggtaacggtgtacatgctaacctccatgtagcgttccaccgttcttcttccgagaaaatccactccaacgagatctctagcgactctatcggtgttctgggttaccagaaaaccgttgaccacaccaaagtgaactccaaactcagcctgttcttcgaaatcaaatct

SEQ ID NO: 3 - DNA, 1623 bp, Artificial Sequence
  misc_signal (1)..(6): NdeI restriction site
  promoter (4)..(6): start codon
  CDS (4)..(1617): CRM197 for expression in E. coli
  terminator (1612)..(1614): stop codon
  terminator (1615)..(1617): stop codon
  misc_signal (1618)..(1623): BamHI restriction site
catatgggtgccgatgacgtggttgactcttccaaaagcttcgtcatggaaaacttcagctcctatcacggcactaaaccgggttatgtcgacagcatccagaaaggcatccagaaaccgaaatctggcactcagggtaactatgacgacgactggaaagagttctactctaccgacaacaaatacgacgcggctggttattctgtggacaacgaaaacccgctgtctggtaaagctggtggtgttgttaaagtgacctacccgggtctgaccaaagttctggctctgaaagtggacaacgccgaaaccatcaaaaaagaactgggtctgtctctgaccgaaccgctgatggaacaggtaggtaccgaggaattcatcaaacgttttggtgatggtgcgtcccgtgttgtactgtctctgccatttgccgaaggttctagctctgtcgagtacatcaacaactgggagcaggccaaagctctgtctgtggaactggaaatcaacttcgagacccgtggtaaacgtggtcaggacgcaatgtatgaatacatggcacaggcttgcgcgggtaaccgtgtacgtcgttctgtaggttcttccctgtcttgcatcaacctggactgggatgtcatccgtgacaaaaccaaaaccaaaatcgagtccctgaaagagcacggtccgatcaaaaacaaaatgagcgaatctccgaacaaaacggtctctgaggaaaaagcgaaacagtacctggaagaattccatcagaccgccctggaacacccggaactgtctgaactgaaaaccgttaccggtactaacccggttttcgcaggtgctaactacgcagcgtgggcggttaacgtagcccaggtaatcgattccgaaaccgcagacaacctggaaaaaacgactgcggctctgtctattctgccgggtattggtagcgtgatgggtattgcagatggtgcagttcaccacaacacggaagaaatcgttgcgcagtctatcgctctgtcttctctgatggtagcacaggcgatcccgctggttggtgaactggttgacattggcttcgcggcctacaacttcgttgaatccatcatcaacctgttccaggttgtgcacaactcttacaaccgtccagcttactctccgggtcacaaaacccagccgttcctgcacgacggttatgcggtttcttggaacaccgttgaagacagcatcatccgtactggtttccagggtgaatctggccacgacatcaaaatcactgctgaaaacaccccgctgccgatcgcaggtgttctcctgccaactattccgggtaaactggacgtgaacaaatccaaaacgcacatctccgtgaacggtcgtaaaatccgcatgcgttgtcgtgcgattgatggtgacgttactttctgtcgtccgaaatctccggtctacgtaggtaacggtgtacatgctaacctccatgtagcgttccaccgttcttcttccgagaaaatccactccaacgagatctctagcgactctatcggtgttctgggttaccagaaaaccgttgaccacaccaaagtgaactccaaactcagcctgttcttcgaaatcaaatcttaatgaggatcc

SEQ ID NO: 4 - protein, 536 aa, Artificial Sequence <22>
  Protein CRM197 encoded by SEQ ID NO: 3
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 5 - DNA, 1704 bp, Artificial Sequence
  misc_signal (1)..(6): NdeI restriction site
  promoter (4)..(6): start codon
  CDS (4)..(87): HisTag
  mat_peptide (4)..(1692): HisTag-CRM197 fusion protein
  misc_signal (72)..(87): CDS for enterokinase cut site
  CDS (88)..(1692): CRM197
  terminator (1693)..(1695): stop codon
  terminator (1696)..(1698): stop codon
  misc_signal (1699)..(1704): BamHI restriction site
catatgggtggttctcatcatcaccatcatcacggcatggcatctatgactggtggtcagcagatgggtcgtgatgacgatgacaaaggtgccgatgacgtggttgactcttccaaaagcttcgtcatggaaaacttcagctcctatcacggcactaaaccgggttatgtcgacagcatccagaaaggcatccagaaaccgaaatctggcactcagggtaactatgacgacgactggaaagagttctactctaccgacaacaaatacgacgcggctggttattctgtggacaacgaaaacccgctgtctggtaaagctggtggtgttgttaaagtgacctacccgggtctgaccaaagttctggctctgaaagtggacaacgccgaaaccatcaaaaaagaactgggtctgtctctgaccgaaccgctgatggaacaggtaggtaccgaggaattcatcaaacgttttggtgatggtgcgtcccgtgttgtactgtctctgccatttgccgaaggttctagctctgtcgagtacatcaacaactgggagcaggccaaagctctgtctgtggaactggaaatcaacttcgagacccgtggtaaacgtggtcaggacgcaatgtatgaatacatggcacaggcttgcgcgggtaaccgtgtacgtcgttctgtaggttcttccctgtcttgcatcaacctggactgggatgtcatccgtgacaaaaccaaaaccaaaatcgagtccctgaaagagcacggtccgatcaaaaacaaaatgagcgaatctccgaacaaaacggtctctgaggaaaaagcgaaacagtacctggaagaattccatcagaccgccctggaacacccggaactgtctgaactgaaaaccgttaccggtactaacccggttttcgcaggtgctaactacgcagcgtgggcggttaacgtagcccaggtaatcgattccgaaaccgcagacaacctggaaaaaacgactgcggctctgtctattctgccgggtattggtagcgtgatgggtattgcagatggtgcagttcaccacaacacggaagaaatcgttgcgcagtctatcgctctgtcttctctgatggtagcacaggcgatcccgctggttggtgaactggttgacattggcttcgcggcctacaacttcgttgaatccatcatcaacctgttccaggttgtgcacaactcttacaaccgtccagcttactctccgggtcacaaaacccagccgttcctgcacgacggttatgcggtttcttggaacaccgttgaagacagcatcatccgtactggtttccagggtgaatctggccacgacatcaaaatcactgctgaaaacaccccgctgccgatcgcaggtgttctcctgccaactattccgggtaaactggacgtgaacaaatccaaaacgcacatctccgtgaacggtcgtaaaatccgcatgcgttgtcgtgcgattgatggtgacgttactttctgtcgtccgaaatctccggtctacgtaggtaacggtgtacatgctaacctccatgtagcgttccaccgttcttcttccgagaaaatccactccaacgagatctctagcgactctatcggtgttctgggttaccagaaaaccgttgaccacaccaaagtgaactccaaactcagcctgttcttcgaaatcaaatcttaatgaggatcc

SEQ ID NO: 6 - protein, 563 aa, Artificial Sequence
  Protein encoded by SEQ ID NO: 5
MGGSHHHHHHGMASMTGGQQMGRDDDDKGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 7 - protein, 535 aa, Escherichia coli
  PEPTIDE (1)..(535): CRM197 after tag removal
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2012/0128727 A1
(43) Pub. Date: May 24, 2012

(54) BACTERIAL EXPRESSION OF AN ARTIFICIAL GENE FOR THE PRODUCTION OF CRM197 AND ITS DERIVATIVES

(76) Inventors: Piero Baglioni, Fiesole (FI) (IT); Alejandro Hochkoeppler, Bologna (IT); Alessandra Stefan, Bologna (IT)

(21) Appl. No.: 13/379,724

(22) PCT Filed: Jun. 25, 2010
(86) PCT No.: PCT/IB10/52910

§ 371 Date: Dec. 21, 2011

(30) Foreign Application Priority Data
Jun. 25, 2009 (IT) FI2009A000137

(51) Int. Cl. A61K 38/45; C12N 15/62; C12N 15/63; A61P 9/10; C12N 9/96; A61P 37/04; A61P 35/00; C12N 15/54; C12N 1/21

(52) U.S. Cl. 424/282.1; 536/23.2; 435/320.1; 435/252.33; 435/188; 424/94.3

(57) ABSTRACT

The present invention relates to polynucleotide sequences comprising the SEQ ID N° 1 encoding CRM197 and optimised for its expression in E. coli. The invention consequently concerns a method for the production of CRM197 in E. coli via a fusion protein CRM197-tag.

### FIELD OF THE INVENTION

The present invention relates to the field of the production of proteins of pharmacological interest by means of artificial gene sequences, said sequences being inserted in expression vectors, the over-expression of the corresponding proteins in micro-organisms converted with said expression vectors, and a method for isolating the proteins expressed; in particular, it relates to the construction of an artificial gene encoding CRM197 as a whole and its derivatives, to the expression of CRM197 and its derivatives in Escherichia coli, and to a method for the isolation and purification of the protein CRM197.

### STATE OF THE ART

The protein CRM197 (cross-reacting material 197, 58 kDa) is a variant of the diphtheria toxin (DTx) characterised by a single mutation that reduces its toxicity, (i.e. the nucleotide variation produces a glycine-glutamic acid substitution in position 52) (Uchida T. et al, 1973; Giannini G. et al, 1984). The protein CRM197 nonetheless retains the same inflammatory and immunostimulant properties as the diphtheria toxin and it is widely used in the preparation of conjugated vaccines against Bordetella pertussis, Clostridium tetani, Corynebacterium diphtheriae, Hepatitis B virus and Haemophilus influenzae type B (WO 93/24148 and WO 97/00697, WO 02/055105). Like the wild-type diphtheria toxin, CRM197 comprises two domains, A and B, bonded together by a disulphide bridge. The A domain (21 kDa) is the catalytic domain, while the B domain (37 kDa) contains one subdomain for bonding to the cell receptor and another subdomain for the translocation (Gill D. M. et al, 1971; Uchida T. et al, 1973). Like DTx, the protein CRM197 is capable of binding (by means of the B domain) to the cell receptor HB-EGF (heparin binding epidermal growth factor), which enables its translocation inside the cell by endocytosis. Exposure to the low pH in the endosome induces a conformational change essential to the insertion of the B domain in the membrane and to the subsequent translocation of the A domain in the cytosol (Papini E. et al, 1993; Cabiaux V. et al, 1997). An essential condition for translocation is the rupture of a peptide bond between the two domains A and B by a protease. Combined with the reduction of the disulphide bridge, this digestion releases the A domain, making it active, while the whole protein, synthesised as a single polypeptide, is inactive (Gill D. M. et al, 1971).

The A domain of the diphtheria toxin has an ADP-ribosylating activity and catalyses the transfer of the ADP-ribose group from the NAD to the elongation factor 2 (EF-2), which is involved in protein synthesis. The complex that forms is inactive and consequently induces an interruption of the eukaryotic protein synthesis (Honjio T. et al, 1971). The cytotoxic effect of the protein is also due to another activity of the A domain, which is capable of non-specifically degrading the DNA (Giannini G. et al, 1984). This endonuclease activity depends on the divalent cations and it is retained in the CRM197 as well (Bruce C. et al, 1990; Lee J. W. et al, 2005).

CRM197 and other non-toxic variants have always been produced using lysogenic cultures of Corynebacterium diphtheriae infected with particular 13 phages whose genome contains a mutated version of the tox gene that encodes the diphtheria toxin (DTx). The diphtheria toxin and the other variants are secreted into the culture medium under particular growth conditions, then recovered by filtering or precipitation, and subsequently purified using chromatographic methods (Cox J., 1975). The procedures initially used for the production of both DTx and its derivatives (CRMs) could not guarantee a high yield, however, so the production of CRM197 from single lysogenic strains of Corynebacterium was not economically advantageous for use as a conjugate in vaccines. To increase the production of CRM197 to an industrial scale, double and triple lysogenic mutants were subsequently isolated, which contain two or three tox genes integrated in the chromosome (Rappuoli R. et al, 1983; Rappuoli R., 1983). In 1990, Rappuoli described a process for the production of proteins derived from DTx that uses a strain of Corynebacterium with two copies of the mutated tox gene integrated in the chromosome. Growth conditions were also established (culture medium, concentration of ferrous ions, growth temperature, percentage of oxygen, etc) to increase the yield (U.S. Pat. No. 4,925,792, 1990). The CRM197 accumulates in the culture medium throughout the logarithmic growth phase, right up to the start of the stationary phase, and it peaks around 20 hours after fermentation has started. There is subsequently evidence of a considerable decline in the yield, however, due probably to proteolysis (U.S. Pat. No. 4,925,792, 1990).

It is important to note that the construction of double and triple lysogenic strains in order to increase expression efficiency is a lengthy process that entails a laborious screening phase. An alternative way to obtain high levels of CRM197 uses a specific plasmid, pPX3511, obtained from the fusion of the phage gene encoding CRM197 with the plasmid pNG-22 (U.S. Pat. No. 5,614,382, 1995). This makes it possible to increase the number of copies of the gene (up to 5-10 per cell) without having to select pluri-lysogenic bacterial strains. Here again, as in the case of the Corynebacterium strains infected by the phage β197^{tox-}, CRM197 is expressed in particular culture media with a low ferrous content. Despite a reduction in the amount of time required for the genetic handling of the bacterial strain, the output of CRM197 does not increase dramatically by comparison with the use of double lysogenes. Fermentation processes for the production of DTx, or various other CRMs, have recently been described in several patents, always involving the use of C. diphtheriae cultures. Generally speaking, growth takes place under controlled conditions of temperature, agitation and aeration, and the maximum production of the toxin and/or its derivatives occurs after 20 hours of culture (Dehottay P. M. H. et al, US2008/0193475; Wolfe H. et al, US2008/0153750).

On the other hand, studies on the use of bacterial hosts as an alternative to Corynebacterium have been limited. Tests have been conducted in Escherichia coli on the expression of the domains A and B, and on some intermediate forms of DTx (the A domain together with portions of the B domain). These studies have generally been conducted to examine in detail the role of the domains A and B (and portions thereof) in terms of toxicity, bonding to the receptor, protein folding and stability (Bishai W R et al, 1987a; Bishai W R et al, 1987b). These fragments, some of which are produced as fusion proteins, have been expressed in Escherichia coli using different promoters and different expression conditions with a view to assessing their solubility and ultimate yield (which varies from 0.4 to 10 mg/L, corresponding to approximately 7% of the total protein). In parallel, a fragment has been cloned of 1875 bp, comprising the original tox promoter, the signal sequence and the whole gene encoding CRM197. Used as a control in Western blotting experiments, this clone seems to be more stable than the various fragments when expressed at periplasmatic level, while the solubility of the protein drops dramatically when expressed in the cytoplasm at high temperature (Bishai W R et al, 1987b).

While it has been possible to express the whole A domain using the natural tox promoter (Leong D. et al, 1983), the expression of the B domain alone in Escherichia coli has proved more complicated because this domain is highly unstable and it is only expressed in fusion with a tag (Spilsberg B. et al, 2005).

Clearly, the heterologous production of the toxin and its derivatives is restricted by numerous problems relating to the adoption of the optimal protein configuration, the potential degradation and the low final yield. One strategy to avoid the formation of the two intramolecular disulphide bridges responsible for the ideal protein configuration involved the construction of several modified peptide derivatives, and particularly the peptide DTa (consisting of the first 185 aa of the CRM197 sequence), the peptide DTb (255 aa, which has a deletion of the domain binding to the cell receptor and of 8 aa at the N-terminal), and the peptide DTaDTb obtained from the fusion of the previous two peptides (440 aa). These fragments have been synthesised by PCR using the C. diphtheriae genome as the template and they were subsequently expressed in E. coli by exploiting the tryptophan induction system (Corvaia N. et al, FR 2827606A1 2003).

There has recently been a growing interest in CRM197 because of its potential antitumour action relating to its capacity to bind the soluble form of HB-EGF (Mekada et al, US 2006/0270600A1). This antitumour function is attributable not only to CRM197, but also to other non-toxic derivatives of the DT toxin (e.g. the double mutant DT52E148K, or the fusion protein GST-DT). These mutants have been constructed by PCR, starting from the gene encoding CRM197. In said studies, however, the whole CRM197 was produced using cultures of C. diphtheriae, grown at 35° C. for 16-17 hours. The CRM197 was purified from the supernatant by means of an initial precipitation with ammonium sulphate, followed by three successive steps in ion exchange and hydrophobic chromatography (Mekada et al, US 2006/0270600A1).

Thus, there are no studies available in the literature that describe the expression of the whole diphtheria toxin, or of CRM197, in E. coli.

Hence the evident need to dispose of an alternative method for the production of CRM197 (and its derivatives) with cost-effective yields in a short space of time and, preferably, by means of the use of alternative bacterial hosts to Corynebacterium.

### DEFINITIONS AND ABBREVIATIONS

CRM197: cross-reacting material
DTx: diphtheria toxin
DTA diphtheria toxin A domain
DTB: diphtheria toxin B domain
EF-2: elongation factor-2
SDS-PAGE: sodium dodecyl sulfate-polyacrylamide gel electrophoresis
IPTG: isopropyl-β-D-thiogalactopyranoside

### SUMMARY OF THE INVENTION

The present invention solves the above-described problems by means of an artificial polynucleotide sequence (SEQ ID N° 1) specific for the over-expression of the protein CRM197 in Escherichia coli. The gene can be associated with a tag sequence and consequently enable the expression in E. coli of a fusion protein, CRM197-tag. The invention also concerns plasmids containing the sequence SEQ ID N° 1 and strains of Escherichia coli genetically modified by the introduction of said plasmids. In one aspect, the invention concerns the recombinant fusion protein CRM197-tag produced from the above-mentioned genetically modified E. coli.

The invention also concerns the process for the production of the recombinant protein CRM197 (domains A and B) with an N-terminal tag by means of its expression in E. coli, genetically modified as explained above, and its subsequent purification. The process also involves the removal of the tag to obtain the protein CRM197 in its native form.

The invention provides a new method for the production of the protein CRM197, and similar proteins, as an alternative to using the micro-organism Corynebacterium diphtheriae. According to the procedure described in the invention, the protein of interest can be obtained in large quantities both for basic research and for applications in the medical-therapeutic field. The invention offers the following advantages: i) it uses a micro-organism, Escherichia coli, that is amply used in the expression of heterologous proteins for industrial and pharmacological applications; ii) the genetics of E. coli have been known for years and numerous alternative systems (vectors and strains) are available for its expression; iii) it is a non-pathogenic micro-organism; iv) the use of E. coli enables the production times to be reduced because it grows rapidly with high biomass yields.

### BRIEF DESCRIPTION OF THE FIGURES

FIG. 1 shows an electrophoretic run (SDS-PAGE 10%) where you can see the band corresponding to the protein with SEQ ID N° 6 (CRM197-tag, 61 kDa) obtained from total protein extracts of different bacterial cultures of E. coli, i.e. BL21AI (lanes 1, 2, 3, 4) and BL21(DE3) (lanes 5, 6, 7, 8). The cultures were submitted to various induction times (1 h, 3 h and overnight). Lane M: standard molecular mass markers; lanes 1 and 5: samples not induced; lanes 2 and 6: samples induced for 1 h; lanes 3 and 7: samples induced for 3 h; lanes 4 and 8: samples induced overnight.

FIG. 2 illustrates the tests conducted on the solubilisation of the protein CRM197-tag from the insoluble fraction. All the tests were conducted using a solution containing urea 6-7 M. Lanes 1 and 2: soluble fraction obtained from non-induced (1) and induced (2) cultures; lane 3: standard molecular mass markers; lane 4: solubilisation solution and Tween 20 at 20° C.; lane 5: solubilisation solution and Triton X-100 at 20° C.; lane 6: solubilisation solution and reducing agent (β-mercaptoethanol 20 mM) at 20° C.; lane 7: solubilisation solution and SDS at 20° C.; lane 8: solubilisation solution and Triton X-100 at 30° C.; lane 9: solubilisation solution and reducing agent at 30° C.

FIG. 3 shows an electrophoretic run of several fractions obtained after affinity chromatography. Lane 1: sample solubilised with urea 6-7 M, pre-column; lane 2: unbonded flow-through in the column; lanes 3 and 4: first fractions eluted with the imidazol gradient; lanes 5-10: fractions corresponding to the central portion of the elution peak.

FIG. 4 shows a SOS-PAGE (10%) gel in which the purification steps are visible. Lane M: standard molecular mass markers; lane 1: soluble fraction; lane 2: total extract solubilised with urea; lane 3: sample after affinity chromatography; lane 4: sample after gel-filtering chromatography.

FIG. 5 shows the electrophoretic run of a sample of CRM197 before and after digestion with enterokinase. M: standard molecular mass markers; lane 1: CRM197-tag not treated with enterokinase; lane 2: CRM197-tag digested at 24° C. for 20 h. The samples were boiled in the presence of a reducing agent. The visible bands correspond to the B domain, the A domain and the A-tag domain (respectively a, b, c).

### DETAILED DESCRIPTION OF THE INVENTION

The sequence corresponding to the whole CRM197 described by Giannini G. et al (1984), without the natural signal sequence for exportation outside the cell, was used to obtain a polynucleotide sequence SEQ ID N° 1 optimised for the expression in E. coli, with the aid of the Leto software (Entelechon GmbH Regensburg, Germany).

The gene sequence SEQ ID N° 1 can also be associated, at both the 5′ and the 3′ ends, with an oligonucleotide sequence that encodes a tag polypeptide to facilitate its cytoplasmic stability and/or subsequent purification using matrices and resins with a high affinity for the various tag peptides. There are numerous known nucleotide sequences that encode tag polypeptides. Among these, there are the nucleotide sequences encoding 6, 8, 10 histidine (H) (His-tag), for the tag MASMTGGQQMG (T7-tag), for NDYKDDDDKC (FLAG-tag), for WSHPQFEK (Strep-tag), for YPYDVPDYA (HAT-tag), for KETAAAKFERQHMDS (S-tag), and for NEQKLISEEDLC (Myc-tag).

The gene SEQ ID N° 1 can also be associated with other tag sequences, e.g. those encoding thioredoxin (Trx), glutathione-S-transferase (GST), maltose-binding protein (MBP), cellulose-binding protein (CBD) and chitin-binding protein (CBP).

Tag sequences can be suitably associated with specific cutting sequences for recognition by suitable enzymes capable of subsequently removing the tag. Enterokinase, thrombin, factor Xa or furin are preferably used to remove the tag, the best-known and most often used cutting peptide sequences of which are DDDDK, LVPRGS, IE/DGR and RXXR, respectively.

In one preferred embodiment, the gene SEQ ID N° 1 is associated with a polynucleotide that encodes a poly-histidine tag. The his-tag sequence can be added at both the 5′-terminal and the 3′-terminal end.

The following are examples of his-tag peptide sequences: MGGSHHHHHHGMASMTGGQQMGR, MGSSHHHHHHSSG, MGSSHHHHHHSSGL, MGSGHHHHHH, MGHHHHHHHHHHSSG, MHHHHHHSSG, ALEHHHHHH, AALEHHHHHH.

One particularly preferred embodiment is the SEQ ID N° 2, where a sequence of 84 nucleotides has been added to the SEQ ID N° 1 sequence at the 5′-terminal end, encoding the sequence containing 6 histidines MGGSHHHHHHGMASMTGGQQMGR and the cutting sequence for enterokinase DDDDK.

Of course, it is for preferable for sequences comprising the SEQ ID N° 1 to be suitable for completing with start and stop codons, and with suitable sequences that encode the recognition sites of the restriction enzymes used for cloning purposes.

Genes comprising the SEQ ID N° 1 can be prepared by chemical synthesis and then cloned in suitable expression vectors. In one particular embodiment, the artificial sequences SEQ ID N° 1 and 2 were prepared synthetically by means of an assembly procedure, obtaining SEQ ID N° 3 and 5, respectively, that encode the proteins with sequences SEQ ID N° 4 and 6, respectively.

The present invention also relates to expression vectors (plasmids) comprising the sequence SEQ ID N° 1 and preferably its derivatives with tags and specific recognition sites for restriction enzymes and/or proteases.

A plasmid from the series pET is preferably used to clone the artificial gene comprising the SEQ ID N° 1. In particular, the vector pET9a contains the promoter T7 specific for the RNA polymerase enzyme of the phage T7. This polymerase is extremely efficient (more so than the bacterial RNA polymerase) and specific (it does not recognize bacterial promoters). In addition to the plasmid pET9a, other vectors in the pET series (Novagen) that are suitable for the process include: pET3a, pET3b, pET3c, pET5a, pET5b, pET5c, pET9b, pET9c, pET12a, pET12b, pET12c, pET17b and, in general, all the vectors that have a strong phage T7 promoter (e.g. pRSETA, B and C [Invitrogen] and pTYB1, pTYB2, pTYB3 and pTYB4 [ New England Biolabs]).

For cloning purposes, it is preferable to use NdeI and BamHI as restriction enzymes.

The resulting construct can be used to convert strains of Escherichia coli. Said E. coli strains can be characterised by alternative gene expression regulating systems that exploit different inductors, such as IPTG (isopropyl-β-D-thiogalactopyranoside) or arabinose.

In the case of pET-type plasmids being used, which contain the promoter T7 specific for the enzyme RNA polymerase of the phage T7, then the E. coli strains suitable for conversion with a pET construct containing the SEQ ID N° 1 may be any of those capable of providing the T7 RNA polymerase enzyme, but preferably: Escherichia coli type B, such as ER2566, ER2833, ER3011, ER3012, BL21AI™, BL21(DE3), BL21Star™(DE3), BL21-Gold(DE3), BL21(DE3)pLys, C41(DE3), C43(DE3), BLR(DE3), B834(DE3 Tuner™(DE3); or Escherichia coli derived from K-12, such as HMS174(DE3), AD494(DE3), Origami™(DE3), NovaBlue(DE3), Rosetta™(DE3). The bacterial strains are preferably converted by electroporation, but other known methods may be equally suitable.

In a particular embodiment, the genes with SEQ ID N° 3 and 5, respectively comprising the SEQ ID N° 1 and 2, were synthesised chemically and then cloned in a particular plasmid of the pET series. The vector used for cloning and expression was the pET9a (Novagen, Darmstadt, Germany) characterised by a replication origin pBR322 that guarantees: a large number of copies per cell; a selective marker to keep the plasmids inside the bacterial host (the kan gene for kanamycin resistance); a polylinker region containing numerous restriction sites suitable for cloning; and a specific promoter inducible to regulate the over-expression of CRM197.

NdeI and BamHI were used as restriction enzymes for the cloning of the artificial gene inside the plasmid (in the polylinker) and sequencing was used to verify its proper orientation and position. The resulting construct was used to convert several strains of E coli by electroporation, selecting the converted colonies on Petri dishes (containing solid LB with added kanamycin). Among the bacterial strains suitable for CRM197 expression cloned in the vector pET9a, two derivatives of Escherichia coli type B were chosen, i.e. BL21AI and BL21(DE3). Both contain a copy of the gene encoding the phage T7 RNA polymerase integrated in the chromosome, controlled by an inducible promoter. Once inside the cell, this enzyme is able to activate the transcription of the artificial gene CRM197 or CRM197-tag cloned downstream from the promoter pT7. The strain BL21AI has the gene encoding the T7 RNA polymerase controlled by the promoter p_{BAD}, so induction takes place thanks to the addition of arabinose to the culture medium. The strain BL21(DE3) was obtained instead thanks to the integration in the bacterial genome of a prophage λ(DE3) containing the gene for the T7 RNA polymerase controlled by the lac promoter. In this latter case, the cascading induction of the expression system is activated by IPTG, a lactose analogue. Other strains of E. cell suitable for conversion with the pET9a-CRM197 construct and for the expression of the protein of interest are the derivatives of BL21(DE3), such as BL21Star™(DE3), BL21-Gold(DE3), BL21(DE3)pLys, the derivatives of ER2566, and all the modified B or K-12 strains containing a copy of the gene encoding the T7 RNA polymerase in their genome.

Once the converted strains of E. coli had been selected, expression tests were conducted in different culture and induction conditions. The object of the preliminary tests was to identify the method enabling high levels of protein CRM197 to be obtained by comparison with the bacterial proteins (preferably up to approximately 30-40%). The factors considered were the culture medium, the growth temperature (30° C. and 37° C.), the concentration of the inducers and the induction time. The culture medium used was the classic LB, but other rich culture media that enable a high biomass production can be used too. When a recombinant protein is over-expressed, the product can be secreted into the medium (if it has a specific signal sequence) or it can build up in the cytoplasm in soluble form, or in the form of insoluble inclusion bodies. The protein's localisation influences the subsequent purification process. In the specific case of the fusion protein CRM197-tag with SEQ ID N° 6, obtained from the transcription of the artificial gene represented by SEQ ID N° 5 (with his-tag), it was found that the protein is expressed by the body in insoluble form (inclusion bodies) and accumulates in a highly convenient manner for the purposes of an industrial production. The expression protocol described in the invention involves the accumulation of CRM197-tag in said insoluble form and describes the steps involved in recovering it in soluble form and renaturing it to obtain the protein in its biologically active form. Moreover, the invention includes two chromatographic purification steps and a final step to remove the tag. The choice of the most suitable chromatographic method depends on the chemical-physical characteristics of the CRM197-tag, such as the pI (isoelectric point), the amino acid composition and the dimensions. Fusion with a tag enables the protein to be purified using a particular resin with a high affinity for the tag (both in the column and in batches). The tag's presence is useful both to increase the stability of the protein in the cytoplasm and for its subsequent purification.

In one aspect, therefore, the invention relates to the recombinant fusion protein CRM197-tag encoded by a polynucleotide comprising the SEQ ID N° 1 and a brief sequence encoding a polypeptide tag.

Particularly preferred is a recombinant fusion protein of sequence SEQ ID N° 6, encoded by a nucleotide comprising the SEQ ID N° 2.

The above-described recombinant fusion protein CRM197-tag is potentially useful for medical purposes, for the treatment of tumours such as cancers of the breast, ovaries and prostate, or for the reduction of atherosclerotic plaques. The aforesaid fusion protein can also be useful as a conjugated carrier for vaccines such as those against Pneumococcus haemophilus influenzae, Meningococcus, Streptococcus pneumoniae and other pathogenetic bacteria.

The invention further concerns the process for producing a CRM197-tag protein, said process comprising the use of E. coli strains modified as explained above.

Said process preferably comprises:
(i). the suitably-induced expression of the protein by means of cultures of E. coli as described above;
(ii). extraction by means of:
   a. lysis in a buffer containing Tris-HCl 20-50 mM pH 7.5-8.5, NaCl 100-150 mM, detergent 0.5-1.5% and protease inhibitor 0.5-1.5%, for 1.5-2.5 hours at 0-5° C., with agitation;
   b. separation of the supernatant from the solid residue (pellet);
   c. treatment of the solid residue resulting from the previous passage with a solubilisation buffer at pH 7.5-8.5 containing Tris-HCl 20-50 mM, NaCl 100-150 mM, detergent 0.5-1.5% and urea 5-7 M, for 1.5-2.5 hours at 20-30° C. with agitation;
   d. separation of the supernatant from the solid residue, the supernatant contains the solubilised CRM197-tag protein;
(iii). purification and renaturing of the protein obtained from step (ii) by:
   a. affinity chromatography or dialysis;
   b. molecular exclusion chromatography (gel filtration) or anion exchange chromatography.

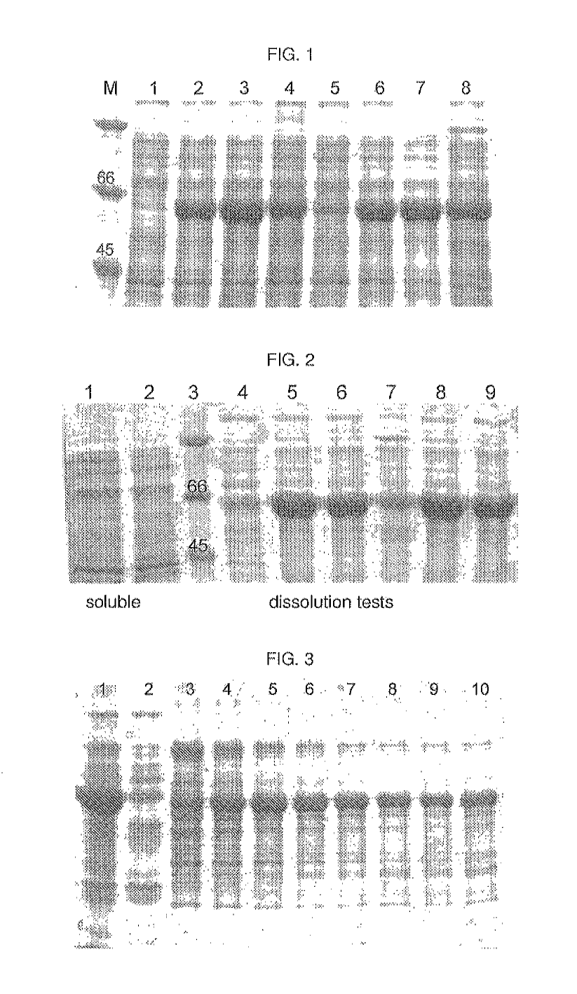

In the embodiment wherein E. coli was modified with a plasmid comprising the SEQ ID N° 2, such as SEQ ID N° 5, the recombinant protein CRM197-tag was produced in fusion with a tag sequence containing 6 histidines that enable its expression and facilitate its subsequent purification by affinity chromatography. The quantity of CRM197 and similar proteins obtained by means of this procedure can be modified by modulating the parameters governing the expression levels (culture medium, growth temperature, induction time, etc). In the case of the E. coli strains of BL21AI or BL21(DE3) converted with the suitable plasmid being used, the best expression conditions are obtained after 3 hours of induction at 37° C. (FIG. 1) and the converted strain BL21AI is preferred. Under these conditions, the expression yield is as high as 40% and the CRM197-tag corresponds to approximately 80% of the insoluble fraction obtained after lysis and removal of the soluble fraction. It is feasible to claim that, in a production process adopting the optimal growth, lysis and recovery conditions, the CRM197-tag expression yield could be as high as 0.5-1 g/L.

In the specific case in which the SEQ ID N° 5 is used, the recombinant CRM197-tag expressing SEQ ID N° 6 has a tag of 28 amino acids containing 6 histidines with a high affinity for divalent metal ions (copper, nickel, etc); this feature is exploited to facilitate the purification of the fusion protein, which is expressed in insoluble form. Affinity chromatography can also be used to remove the denaturing agent needed to recover the CRM197-tag from the insoluble fraction. In this case, the removal takes place gradually (in two inverse-gradient stages) to facilitate the adoption of the correct protein configuration (folding).

The contaminating proteins that have remained associated with the protein of interest can subsequently be removed by gel-filtration chromatography in the case of the molecular masses differing considerably from one another. Alternatively, exploiting the pI value of CRM197 (5.8-5.9), a second purification passage can be conducted using ion exchange chromatography. The invention thus involves two different purification methods subsequent to the affinity chromatography, to be used as appropriate. The final yield of recombinant protein and the purity levels are comparable, whichever type of process is used. The proteins are quantified by Bradford assay and visualised in 10% acrylamide gel (SDS-PAGE). The expression yield of the CRM197-tag protein obtained according to the protocol described in the invention is 250±50 mg/L of culture medium (in graduated flasks with LB medium). As mentioned previously, in an industrial process conducted in a fermenter, using suitable growth media and conditions, the yield increases further. It is worth emphasizing that the method of lysis and extraction described in the invention is simple and inexpensive; moreover, the phases of the process have been designed so as to avoid the need for particular, buffers/reagents or special laboratory equipment (such as the sonicator for cell lysis), all with a view to achieving a protocol suitable for an industrial process.

Finally, the invention concerns the procedure for removing the tag that has had a dual purpose, i.e. to enable the expression of the CRM197, increasing its stability and facilitating its purification.

The invention consequently also concerns a process for the preparation of CRM197, said process being characterised by the use of E. coli strains modified as explained above.

The above-described process for the production of CRM197 preferably involves the expression of the fusion protein CRM197-tag as described above and the subsequent removal of the tag by digestion with a suitable enzyme.

In the case of CRM197-tag with the sequence SEQ ID N° 6, the enzyme suitable for removing the tag is enterokinase and its digestion is preferably conducted at 20-25° C. for 18-24 hours in a buffer containing Tris-HCl 10-20 mM, pH 7.5-8.5, NaCl 40-60 mM, CaCl₂ 1.5-2.5 mM and enzyme at a concentration in the range of 0.01-0.03% weight to weight (w/w).

After the tag has been removed, the protein without the tag is preferably purified by affinity chromatography.

The CRM197 recombinant protein SEQ ID N° 7 obtained by means of the method according to the present invention is identical in structure and function to the CRM197 produced using the known methods; it is obtained in native form and is consequently active, and it can therefore be used for the known applications.

The present invention may be easier to understand in the light of the following examples of embodiments.

SEQUENCES

SEQ ID N^{o} 1 - Artificial sequence encoding CRM197
optimised for expression in E. coli
GGTGCCGAT GACGTGGTTG ACTCTTCCAA AAGCTTCGTC
ATGGAAAACT TCAGCTCCTA TCACGGCACT AAACCGGGTT
ATGTCGACAG CATCCAGAAA GGCATCCAGA AACCGAAATC
TGGCACTCAG GGTAACTATG ACGACGACTG GAAAGAGTTC
TACTCTACCG ACAACAAATA CGACGCGGCT GGTTATTCTG
TGGACAACGA AAACCCGCTG TCTGGTAAAG CTGGTGGTGT
TGTTAAAGTG ACCTACCCGG GTCTGACCAA AGTTCTGGCT
CTGAAAGTGG ACAACGCCGA AACCATCAAA AAAGAACTGG
GTCTGTCTCT GACCGAACCG CTGATGGAAC AGGTAGGTAC
CGAGGAATTC ATCAAACGTT TTGGTGATGG TGCGTCCCGT
GTTGTACTGT CTCTGCCATT TGCCGAAGGT TCTAGCTCTG
TCGAGTACAT CAACAACTGG GAGCAGGCCA AAGCTCTGTC
TGTGGAACTG GAAATCAACT TCGAGACCCG TGGTAAACGT
GGTCAGGACG CAATGTATGA ATACATGGCA CAGGCTTGCG
CGGGTAACCG TGTACGTCGT TCTGTAGGTT CTTCCCTGTC
TTGCATCAAC CTGGACTGGG ATGTCATCCG TGACAAAACC
AAAACCAAAA TCGAGTCCCT GAAAGAGCAC GGTCCGATCA
AAAACAAAAT GAGCGAATCT CCGAACAAAA CGGTCTCTGA
GGAAAAAGCG AAACAGTACC TGGAAGAATT CCATCAGACC
GCCCTGGAAC ACCCGGAACT GTCTGAACTG AAAACCGTTA
CCGGTACTAA CCCGGTTTTC GCAGGTGCTA ACTACGCAGC
GTGGGCGGTT AACGTAGCCC AGGTAATCGA TTCCGAAACC
GCAGACAACC TGGAAAAAAC GACTGCGGCT CTGTCTATTC
TGCCGGGTAT TGGTAGCGTG ATGGGTATTG CAGATGGTGC
AGTTCACCAC AACACGGAAG AAATCGTTGC GCAGTCTATC
GCTCTGTCTT CTCTGATGGT AGCACAGGCG ATCCCGCTGG
TTGGTGAACT GGTTGACATT GGCTTCGCGG CCTACAACTT
CGTTGAATCC ATCATCAACC TGTTCCAGGT TGTGCACAAC
TCTTACAACC GTCCAGCTTA CTCTCCGGGT CACAAAACCC
AGCCGTTCCT GCACGACGGT TATGCGGTTT CTTGGAACAC
CGTTGAAGAC AGCATCATCC GTACTGGTTT CCAGGGTGAA
TCTGGCCACG ACATCAAAAT CACTGCTGAA AACACCCCGC
TGCCGATCGC AGGTGTTCTC CTGCCAACTA TTCCGGGTAA
ACTGGACGTG AACAAATCCA AAACGCACAT CTCCGTGAAC
GGTCGTAAAA TCCGCATGCG TTGTCGTGCG ATTGATGGTG
ACGTTACTTT CTGTCGTCCG AAATCTCCGG TCTACGTAGG
TAACGGTGTA CATGCTAACC TCCATGTAGC GTTCCACCGT
TCTTCTTCCG AGAAAATCCA CTCCAACGAG ATCTCTAGCG
ACTCTATCGG TGTTCTGGGT TACCAGAAAA CCGTTGACCA
CACCAAAGTG AACTCCAAAC TCAGCCTGTT CTTCGAAATC
AAATCT
SEQ ID N^{o} 2 - Artificial sequence encoding
CRM197-HisTag in E. coli
ATGGGTG GTTCTCATCA TCACCATCAT CACGGCATGG
CATCTATGAC TGGTGGTCAG CAGATGGGTC GTGATGACGA
TGACAAA GGT GCCGATGACG TGGTTGACTC TTCCAAAAGC
TTCGTCATGG AAAACTTCAG CTCCTATCAC GGCACTAAAC
CGGGTTATGT CGACAGCATC CAGAAAGGCA TCCAGAAACC
GAAATCTGGC ACTCAGGGTA ACTATGACGA CGACTGGAAA
GAGTTCTACT CTACCGACAA CAAATACGAC GCGGCTGGTT
ATTCTGTGGA CAACGAAAAC CCGCTGTCTG GTAAAGCTGG
TGGTGTTGTT AAAGTGACCT ACCCGGGTCT GACCAAAGTT
CTGGCTCTGA AAGTGGACAA CGCCGAAACC ATCAAAAAAG
AACTGGGTCT GTCTCTGACC GAACCGCTGA TGGAACAGGT
AGGTACCGAG GAATTCATCA AACGTTTTGG TGATGGTGCG
TCCCGTGTTG TACTGTCTCT GCCATTTGCC GAAGGTTCTA
GCTCTGTCGA GTACATCAAC AACTGGGAGC AGGCCAAAGC
TCTGTCTGTG GAACTGGAAA TCAACTTCGA GACCCGTGGT
AAACGTGGTC AGGACGCAAT GTATGAATAC ATGGCACAGG
CTTGCGCGGG TAACCGTGTA CGTCGTTCTG TAGGTTCTTC
CCTGTCTTGC ATCAACCTGG ACTGGGATGT CATCCGTGAC
AAAACCAAAA CCAAAATCGA GTCCCTGAAA GAGCACGGTC
CGATCAAAAA CAAAATGAGC GAATCTCCGA ACAAAACGGT
CTCTGAGGAA AAAGCGAAAC AGTACCTGGA AGAATTCCAT
CAGACCGCCC TGGAACACCC GGAACTGTCT GAACTGAAAA
CCGTTACCGG TACTAACCCG GTTTTCGCAG GTGCTAACTA
CGCAGCGTGG GCGGTTAACG TAGCCCAGGT AATCGATTCC
GAAACCGCAG ACAACCTGGA AAAAACGACT GCGGCTCTGT
CTATTCTGCC GGGTATTGGT AGCGTGATGG GTATTGCAGA
TGGTGCAGTT CACCACAACA CGGAAGAAAT CGTTGCGCAG
TCTATCGCTC TGTCTTCTCT GATGGTAGCA CAGGCGATCC
CGCTGGTTGG TGAACTGGTT GACATTGGCT TCGCGGCCTA
CAACTTCGTT GAATCCATCA TCAACCTGTT CCAGGTTGTG
CACAACTCTT ACAACCGTCC AGCTTACTCT CCGGGTCACA
AAACCCAGCC GTTCCTGCAC GACGGTTATG CGGTTTCTTG
GAACACCGTT GAAGACAGCA TCATCCGTAC TGGTTTCCAG
GGTGAATCTG GCCACGACAT CAAAATCACT GCTGAAAACA
CCCCGCTGCC GATCGCAGGT GTTCTCCTGC CAACTATTCC
GGGTAAACTG GACGTGAACA AATCCAAAAC GCACATCTCC
GTGAACGGTC GTAAAATCCG CATGCGTTGT CGTGCGATTG
ATGGTGACGT TACTTTCTGT CGTCCGAAAT CTCCGGTCTA
CGTAGGTAAC GGTGTACATG CTAACCTCCA TGTAGCGTTC
CACCGTTCTT CTTCCGAGAA AATCCACTCC AACGAGATCT
CTAGCGACTC TATCGGTGTT CTGGGTTACC AGAAAACCGT
TGACCACACC AAAGTGAACT CCAAACTCAG CCTGTTCTTC
GAAATCAAAT CT
Underscored: the sequence encoding the tag
peptide containing 6 histidines
In italics and underscored: 15 nucleotides
that encode the 5 aa recognized by enterokinase
(DDDDK)
SEQ ID N^{o} 3 - Artificial sequence for CRM197
protein expression in E. coli
CATATGGGT GCCGATGACG TGGTTGACTC TTCCAAAAGC
TTCGTCATGG AAAACTTCAG CTCCTATCAC GGCACTAAAC
CGGGTTATGT CGACAGCATC CAGAAAGGCA TCCAGAAACC
GAAATCTGGC ACTCAGGGTA ACTATGACGA CGACTGGAAA
GAGTTCTACT CTACCGACAA CAAATACGAC GCGGCTGGTT
ATTCTGTGGA CAACGAAAAC CCGCTGTCTG GTAAAGCTGG
TGGTGTTGTT AAAGTGACCT ACCCGGGTCT GACCAAAGTT
CTGGCTCTGA AAGTGGACAA CGCCGAAACC ATCAAAAAAG
AACTGGGTCT GTCTCTGACC GAACCGCTGA TGGAACAGGT
AGGTACCGAG GAATTCATCA AACGTTTTGG TGATGGTGCG
TCCCGTGTTG TACTGTCTCT GCCATTTGCC GAAGGTTCTA
GCTCTGTCGA GTACATCAAC AACTGGGAGC AGGCCAAAGC
TCTGTCTGTG GAACTGGAAA TCAACTTCGA GACCCGTGGT
AAACGTGGTC AGGACGCAAT GTATGAATAC ATGGCACAGG
CTTGCGCGGG TAACCGTGTA CGTCGTTCTG TAGGTTCTTC
CCTGTCTTGC ATCAACCTGG ACTGGGATGT CATCCGTGAC
AAAACCAAAA CCAAAATCGA GTCCCTGAAA GAGCACGGTC
CGATCAAAAA CAAAATGAGC GAATCTCCGA ACAAAACGGT
CTCTGAGGAA AAAGCGAAAC AGTACCTGGA AGAATTCCAT
CAGACCGCCC TGGAACACCC GGAACTGTCT GAACTGAAAA
CCGTTACCGG TACTAACCCG GTTTTCGCAG GTGCTAACTA
CGCAGCGTGG GCGGTTAACG TAGCCCAGGT AATCGATTCC
GAAACCGCAG ACAACCTGGA AAAAACGACT GCGGCTCTGT
CTATTCTGCC GGGTATTGGT AGCGTGATGG GTATTGCAGA
TGGTGCAGTT CACCACAACA CGGAAGAAAT CGTTGCGCAG
TCTATCGCTC TGTCTTCTCT GATGGTAGCA CAGGCGATCC
CGCTGGTTGG TGAACTGGTT GACATTGGCT TCGCGGCCTA
CAACTTCGTT GAATCCATCA TCAACCTGTT CCAGGTTGTG
CACAACTCTT ACAACCGTCC AGCTTACTCT CCGGGTCACA
AAACCCAGCC GTTCCTGCAC GACGGTTATG CGGTTTCTTG
GAACACCGTT GAAGACAGCA TCATCCGTAC TGGTTTCCAG
GGTGAATCTG GCCACGACAT CAAAATCACT GCTGAAAACA
CCCCGCTGCC GATCGCAGGT GTTCTCCTGC CAACTATTCC
GGGTAAACTG GACGTGAACA AATCCAAAAC GCACATCTCC
GTGAACGGTC GTAAAATCCG CATGCGTTGT CGTGCGATTG
ATGGTGACGT TACTTTCTGT CGTCCGAAAT CTCCGGTCTA
CGTAGGTAAC GGTGTACATG CTAACCTCCA TGTAGCGTTC
CACCGTTCTT CTTCCGAGAA AATCCACTCC AACGAGATCT
CTAGCGACTC TATCGGTGTT CTGGGTTACC AGAAAACCGT
TGACCACACC AAAGTGAACT CCAAACTCAG CCTGTTCTTC
GAAATCAAAT CTTAATGAGG ATCC
In bold type: the NdeI (CATATG) and BamHI
(GGATCC) restriction sites
Underscored: the start (ATG) and stop (TAA TGA)
codons.
SEQ ID N^{o} 4 - CRM197 protein sequence from
SEQ ID N^{o} 3
MGADDVVDSS KSFVMENFSS YHGTKPGYVD SIQKGIQKPK
SGTQGNYDDD WKEFYSTDNK YDAAGYSVDN ENPLSGKAGG
VVKVTYPGLT KVLALKVDNA ETIKKELGLS LTEPLMEQVG
TEEFIKRFGD GASRVVLSLP FAEGSSSVEY INNWEQAKAL
SVELEINFET RGKRGQDAMY EYMAQACAGN RVRRSVGSSL
SCINLDWDVI RDKTKTKIES LKEHGPIKNK MSESPNKTVS
EEKAKQYLEE FHQTALEHPE LSELKTVTGT NPVFAGANYA
AWAVNVAQVI DSETADNLEK TTAALSILPG IGSVMGIADG
AVHHNTEEIV AQSIALSSLM VAQAIPLVGE LVDIGFAAYN
FVESIINLFQ VVHNSYNRPA YSPGHKTQPF LHDGYAVSWN
TVEDSIIRTG FQGESGHDIK ITAENTPLPI AGVLLPTIPG
KLDVNKSKTH ISVNGRKIRM RCRAIDGDVT FCRPKSPVYV
GNGVHANLHV AFHRSSSEKI HSNEISSDSI GVLGYQKTVD
HTKVNSKLSL FFEIKS
SEQ ID N^{o} 5 - Artificial sequence for the
expression of the fusion protein CRM197-HisTag
in E. coli
CATATGGGTG GTTCTCATCA TCACCATCAT CACGGCATGG
CATCTATGAC TGGTGGTCAG CAGATGGGTC GTGATGACGA
TGACAAAGGT GCCGATGACG TGGTTGACTC TTCCAAAAGC
TTCGTCATGG AAAACTTCAG CTCCTATCAC GGCACTAAAC
CGGGTTATGT CGACAGCATC CAGAAAGGCA TCCAGAAACC
GAAATCTGGC ACTCAGGGTA ACTATGACGA CGACTGGAAA
GAGTTCTACT CTACCGACAA CAAATACGAC GCGGCTGGTT
ATTCTGTGGA CAACGAAAAC CCGCTGTCTG GTAAAGCTGG
TGGTGTTGTT AAAGTGACCT ACCCGGGTCT GACCAAAGTT
CTGGCTCTGA AAGTGGACAA CGCCGAAACC ATCAAAAAAG
AACTGGGTCT GTCTCTGACC GAACCGCTGA TGGAACAGGT
AGGTACCGAG GAATTCATCA AACGTTTTGG TGATGGTGCG
TCCCGTGTTG TACTGTCTCT GCCATTTGCC GAAGGTTCTA
GCTCTGTCGA GTACATCAAC AACTGGGAGC AGGCCAAAGC
TCTGTCTGTG GAACTGGAAA TCAACTTCGA GACCCGTGGT
AAACGTGGTC AGGACGCAAT GTATGAATAC ATGGCACAGG
CTTGCGCGGG TAACCGTGTA CGTCGTTCTG TAGGTTCTTC
CCTGTCTTGC ATCAACCTGG ACTGGGATGT CATCCGTGAC
AAAACCAAAA CCAAAATCGA GTCCCTGAAA GAGCACGGTC
CGATCAAAAA CAAAATGAGC GAATCTCCGA ACAAAACGGT
CTCTGAGGAA AAAGCGAAAC AGTACCTGGA AGAATTCCAT
CAGACCGCCC TGGAACACCC GGAACTGTCT GAACTGAAAA
CCGTTACCGG TACTAACCCG GTTTTCGCAG GTGCTAACTA
CGCAGCGTGG GCGGTTAACG TAGCCCAGGT AATCGATTCC
GAAACCGCAG ACAACCTGGA AAAAACGACT GCGGCTCTGT
CTATTCTGCC GGGTATTGGT AGCGTGATGG GTATTGCAGA
TGGTGCAGTT CACCACAACA CGGAAGAAAT CGTTGCGCAG
TCTATCGCTC TGTCTTCTCT GATGGTAGCA CAGGCGATCC
CGCTGGTTGG TGAACTGGTT GACATTGGCT TCGCGGCCTA
CAACTTCGTT GAATCCATCA TCAACCTGTT CCAGGTTGTG
CACAACTCTT ACAACCGTCC AGCTTACTCT CCGGGTCACA
AAACCCAGCC GTTCCTGCAC GACGGTTATG CGGTTTCTTG
GAACACCGTT GAAGACAGCA TCATCCGTAC TGGTTTCCAG
GGTGAATCTG GCCACGACAT CAAAATCACT GCTGAAAACA
CCCCGCTGCC GATCGCAGGT GTTCTCCTGC CAACTATTCC
GGGTAAACTG GACGTGAACA AATCCAAAAC GCACATCTCC
GTGAACGGTC GTAAAATCCG CATGCGTTGT CGTGCGATTG
ATGGTGACGT TACTTTCTGT CGTCCGAAAT CTCCGGTCTA
CGTAGGTAAC GGTGTACATG CTAACCTCCA TGTAGCGTTC
CACCGTTCTT CTTCCGAGAA AATCCACTCC AACGAGATCT
CTAGCGACTC TATCGGTGTT CTGGGTTACC AGAAAACCGT
TGACCACACC AAAGTGAACT CCAAACTCAG CCTGTTCTTC
GAAATCAAAT CTTAATGA GGATCC
In bold type: the NdeI (CATATG) and BamHI (GGATCC)
restriction sites.
Underscored: the 84 nucleotides that encode the
tag peptide containing 6 histidines:
ATGGGTG GTTCTCATCA TCACCATCAT CACGGCATGG
CATCTATGAC TGGTGGTCAG CAGATGGGTC GTGATGACGA
TGACAAA
In italics and underscored: 15 nucleotides
encoding the 5 aa recognized by enterokinase
(DDDDK).
Start codon: ATG
Stop codons: TAA TGA
SEQ ID N^{o} 6 - Protein sequence CRM197-HisTag from
SEQ ID N^{o} 5
MGGSHHHHHH GMASMTGGQQ MGRDDDDK GADDVVDSSK
SFVMENFSSY HGTKPGYVDS IQKGIQKPKS GTQGNYDDDW
KEFYSTDNKY DAAGYSVDNE NPLSGKAGGV VKVTYPGLTK
VLALKVDNAE TIKKELGLSL TEPLMEQVGT EEFIKRFGDG
ASRVVLSLPF AEGSSSVEYI NNWEQAKALS VELEINFETR
GKRGQDAMYE YMAQACAGNR VRRSVGSSLS CINLDWDVIR
DKTKTKIESL KEHGPIKNKM SESPNKTVSE EKAKQYLEEF
HQTALEHPEL SELKTVTGTN PVFAGANYAA WAVNVAQVID
SETADNLEKT TAALSILPGI GSVMGIADGA VHHNTEEIVA
QSIALSSLMV AQAIPLVGEL VDIGFAAYNF VESIINLFQV
VHNSYNRPAY SPGHKTQPFL HDGYAVSWNT VEDSIIRTGF
QGESGHDIKI TAENTPLPIA GVLLPTIPGK LDVNKSKTHI
SVNGRKIRMR CRAIDGDVTF CRPKSPVYVG NGVHANLHVA
FHRSSSEKIH SNEISSDSIG VLGYQKTVDH TKVNSKLSLF
FEIKS
In bold type: the tag sequence (28 amino acids)
containing the 6 histidines (H) and the cutting
site for enterokinase (DDDDK).
SEQ ID N^{o} 7 - CRM197 protein sequence after
removal of the tag from SEQ ID N^{o} 6
GADDVVDSSK SFVMENFSSY HGTKPGYVDS IQKGIQKPKS
GTQGNYDDDW KEFYSTDNKY DAAGYSVDNE NPLSGKAGGV
VKVTYPGLTK VLALKVDNAE TIKKELGLSL TEPLMEQVGT
EEFIKRFGDG ASRVVLSLPF AEGSSSVEYI NNWEQAKALS
VELEINFETR GKRGQDAMYE YMAQACAGNR VRRSVGSSLS
CINLDWDVIR DKTKTKIESL KEHGPIKNKM SESPNKTVSE
EKAKQYLEEF HQTALEHPEL SELKTVTGTN PVFAGANYAA
WAVNVAQVID SETADNLEKT TAALSILPGI GSVMGIADGA
VHHNTEEIVA QSIALSSLMV AQAIPLVGEL VDIGFAAYNF
VESIINLFQV VHNSYNRPAY SPGHKTQPFL HDGYAVSWNT
VEDSIIRTGF QGESGHDIKI TAENTPLPIA GVLLPTIPGK
LDVNKSKTHI SVNGRKIRMR CRAIDGDVTF CRPKSPVYVG
NGVHANLHVA FHRSSSEKIH SNEISSDSIG VLGYQKTVDH
TKVNSKLSLF FEIKS

### EXPERIMENTAL PART

### Example 1

### Synthesis of the Genes SEQ ID N° 3 and SEQ ID N° 4 and Preparation of the Construct pET9a-CRM197-Tag

The synthetic genes were obtained by binding together oligonucleotide multiples of approximately 27-43 bp (with regions overlapping by 10-15 bp). This procedure is called “assembly”. In particular, the various synthetic oligonucleotides were phosphorylated at the ends to enable the binding reaction and then they were mixed in equimolar quantities in the presence of the enzyme Taq DNA ligase. Said enzyme is active at high temperatures (45-65° C.) and catalyses the formation of phosphodiester bonds between the phosphate at position 5′ of one oligonucleotide and the hydroxyl group at position 3′ of another oligonucleotide. The binding product was then amplified by PCR and cloned in the pET9a vector using the NdeI and BamHI enzyme. The primers used for amplification were as follows:

CRM197 fwd:
5′ ggaattCATATGGGTGCCGATGACGTGGTTGA 3′
CRM197 rev:
5′ cgGGATCCTCATTAAGATTTGATTTCGAAG 3′
CRM197-His fwd:
5′ ggaattCATATGGGTGGTTCTCATCATCACCATCA 3′
CRM197-His rev:
5′ cgGGATCCTCATTAAGATTTGATTTCGAAGAACAGG 3′

The PCR (30 cycles) was conducted according to standard protocols using the following quantities:

3 μl binding product
5 μl dNTPs (4 mM)
5 μl 1ThermoPol reaction buffer 10× (New England Biolabs)
2 μl fwd_primer (50 pmol)
2 μl rev_primer (50 pmol)
0.5 μl Vent DNA polymerase (New England Biolabs)
and adding 32.5 μl of water to make up to a volume of 50 μl.

The PCR products comprising the SEQ ID N° 1 and N° 2 were purified to remove the primers, the dNTPs and the enzyme, then digested with NdeI and BamHI, thus obtaining the genes of sequences SEQ ID N° 3 and 5. In parallel, 1 μg of the plasmid pET9a was digested with the same enzymes under the same conditions (37° C. for 2 hours). Finally, the binding reaction was conducted at 16° C. for 12-16 hours using an insert to vector ratio of 1:1 and 3:1. An aliquot of this reaction was used to convert the recipient bacterial cells.

### Example 2

### Bacterial Strains and Culture Media

The BL21AI (Invitrogen) and BL21(DE3) E. coli strains (Novagen) were used as hosts for the expression of CRM197-tag (SEQ ID N° 5). The liquid and solid culture medium generally used was the classic LB (Luria-Bertani; Sambrook et al, 1989, Molecular Cloning: a Laboratory Manual, Cold Spring Harbor Laboratory Press, NY). The suitably-treated host strains were converted using 10 ng of the pET9a-CRM197-tag construct (obtained from example 1); electroporation was conducted according to a standard protocol using suitable 1 mm cuvettes and a pulse of 1.8 kV (Gene Puiser II, Bio-Rad). The electroporated cells were grown for 45 minutes in SOC medium (Sambrook et al, 1989) at 37° C. with agitation, then transferred to a solid LB medium to which kanamycin was added (in a final concentration of 50 μg/mL) to select the transformants. The cultures were generally performed in aerobic conditions at 37° C. with agitation (180 rpm).

### Example 3

### Expression

Arabinose 13 mM (for the BL21AI strain) and IPTG 1 mM (for the BL21[DE3] strain) were added to the culture medium to induce the expression of CRM197-tag SEQ ID N° 5. After selecting the converted strains, expression tests were performed on small volumes (10 mL). Single colonies were grown in 1 mL of LB medium (with kanamycin) and suitably relaunched in fresh medium until the exponential growth phase was reached (confirmed by measuring the spectrophotometric absorbance at 600 nm). The inducers were added at absorbance values of approximately 0.5-0.6 OD and the cultures were induced for various times (1 h, 3 h and 15 h). The cells were collected by centrifugation (4000 g for 15 min) and the resulting cell pellets were lysed to release the total protein. Initially, lysis was done simply by boiling the samples for 5 minutes in the presence of sample buffer solution (Bio-Rad) and 204 of each sample were separated in SDS-PAGE electrophoresis (10% acrylamide). The gels were stained with a solution of Comassie brilliant blue to visualise the protein bands and a band of over-expression corresponding to the CRM197-tag (approximately 61 kDa; FIG. 1) was identifiable. Said band represented approximately 40% of the total proteins visible in the acrylamide gel.

After verifying the expression of the protein of interest, tests were subsequently performed with larger quantities of culture (500 mL) and in optimal conditions (induction for 3 h with arabinose 13 mM).

### Example 4

### Extraction

To lyse the cells without resorting to the use of the sonicator, different lytic solutions of known composition were prepared and their efficacy was assessed, also varying the ratio of the volume of solution to that of the sample. The components of the lysis buffer were: Tris-HCl pH 8 (at a concentration in the range of 20-50 mM), NaCl (at a concentration in the range of 100-150 mM), a detergent at a concentration in the range of 0.5-1.5% (Triton X-100, SDS, Tween 20) and a protease inhibitor (e.g. PMSF 1 mM). We also evaluated the effects of a reducing agent such as β-mercaptoethanol or DTT (10-50 mM). The cell pellets were lysed with agitation for 2 hours on ice. The supernatant (corresponding to the soluble protein fraction) was separated by centrifugation (10,000 g for 30 min) and analysed in SDS-PAGE gel (FIG. 2). The recombinant protein was not visible in this fraction because it accumulates in the form of inclusion bodies and is clustered in the pellet obtained after lysis. The invention consequently involves the use of a solubilisation solution to recover the CRM197-tag from the insoluble fraction (FIG. 2). The components of this solution were: Tris-HCl pH 8 (at a concentration in the range of 20-50 mM), NaCl (at a concentration in the range of 100-150 mM), a detergent 0.5-1.5% (Triton X-100, SDS, Tween 20) and urea 6-7 M. The pellets containing the inclusion bodies were solubilised for two hours with agitation at a temperature in the range of 20-30° C. The supernatant was recovered by centrifugation and analysed in SDS-PAGE gel, where the band corresponding to the CRM197-tag was visible (FIG. 2). In the sample solubilised with urea, the band relating to the CRM197-tag corresponded to approximately 50% of the proteins contained in the gel.

### Example 5

### Purification

The sample solubilised with urea (stored at 4° C.) underwent affinity chromatography (HiTrap Chelating, GE Healthcare) for the dual purpose of a preliminary purification and to remove the urea in order to renature the protein in the column. Another suitable renaturing method is dialysis, using a solution with decreasing concentrations of urea (from 6-7 M to 0 M). The chromatographic column was conditioned and treated according to the manufacturer's instructions. In the case of the CRM197 protein with the 6-histidine tag, the column was complexed with nickel ions (NiSO₄ 0.1M). This procedure includes three stages: 1) removal of the detergent; 2) removal of the urea by means of a two-stage inverse gradient; 3) elution with an imidazole gradient (0-500 mM). The sample was loaded and renatured under slow flow conditions (0.5 mL/min), while the other stages were completed at the flow rate of 1 mL/min. The final fractions obtained contained the CRM197 protein (fused with the tag) in a solution of Tris-HCl pH 8, NaCl, imidazole (FIG. 3 shows some of the chromatographic fractions).

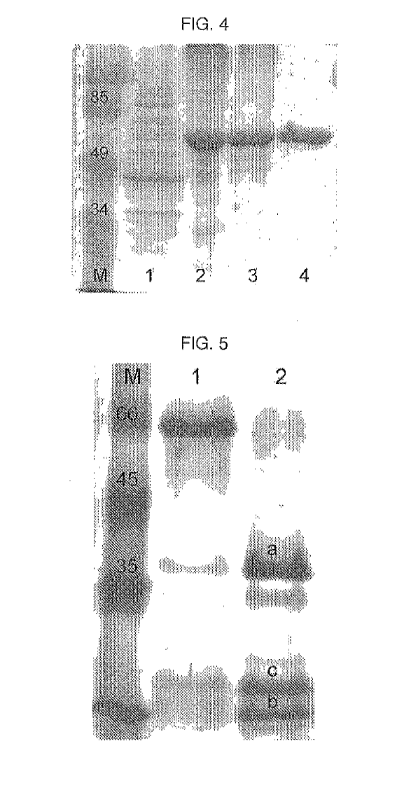

The invention includes a subsequent purification by gel-filtration chromatography (Superdex 200 column, GE Healthcare). Before this step was completed, the sample was concentrated by ultrafiltration (Amicon, Millipore) and desalted to remove the imidazole (HiTrap desalting column, GE Healthcare). The Superdex column was conditioned with buffer containing Tris-HCl 50 mM pH 8, NaCl 150 mM. The fractions were analysed in SDS-PAGE gel and those containing the pure CRM197-tag were pooled and frozen. FIG. 4 shows the various stages of CRM197-tag purification.

As an alternative to molecular exclusion chromatography, the CRM197-tag can be purified by ion exchange chromatography. In this case, it is preferable to use an anion exchange resin conditioned with a suitable buffer at a pH 8.

### Example 6

### Tag Removal

In addition to the 6 histidines needed for purification, the tag sequence (MGGSHHHHHHGMASMTGGQQMGRDDDDK) also contains a cutting site recognized by a specific protease, enterokinase (New England BioLabs), DDDDK.

To obtain the pure recombinant protein without the tag (SEQ ID N° 6), the CRM197-tag (SEQ ID N° 5) was incubated with enterokinase. The digestion reaction was conducted at 22-24° C. for 18-24 h in a buffer of Tris-HCl 20 mM pH 8, NaCl 50 mM, CaCl₂ 2 mM, using a quantity of enzyme corresponding to 0.02% (w/w). FIG. 5 shows a SDS-PAGE gel in which the digested CRM197 is visible (in lane 2) separated into the two domains A and B (the sample was boiled with a reducing agent that disrupts the disulphide bridge between the domains). The protocol involves a subsequent step to separate the CRM197 (without the tag, SEQ ID N° 6) from the tag alone by affinity chromatography (in the same column and using the same resin as was used for the above-described purification of the CRM197-tag).

### REFERENCES

Uchida T., Pappenheimer A. M. Jr, and Greany R., 1973. J Biol Chem, 248:3838-44
Gill D. M., and Pappenheimer A. M. Jr, 1971. J. Biol Chem, 246:1492-1495.
Uchida T., Pappenheimer A. M. Jr, Harper A. A., 1973. J Biol Chem, 248:3845-50.
Papini E., Rappuoli R., Murgia M., and Montecucco C., 1993. J. Biol. Chem., 268:1567-1574.
Cabiaux V., Wolff C., and Ruysschaert J. M., 1997. Int J Biol Macromol, 21:285-98.
Honjo T., Nishizuka Y., Kato I., and Hayaishi O., 1971. J Biol Chem, 246:4251-60.
Giannini G., Rappuoli R., and Ratti G., 1984. Nucleic Acids Res, 12: 4063-4069.
Bruce C., Baldwin R. L., Lessnick S. L., and Wisnieski B. J., 1990. Proc. Natl. Acad. Sci. USA, 87:2995-2998.
Lee J. W., Nakamura L. T., Chang M. P., and Wisnieski B. J., 2005. BBActa, 1747:121-131.
Cox J. C., 1975. Applied Microbiol, 29:464-468.
Rappuoli R., 1983. Applied Envirom Microbiol, 46:560-564.
Rappuoi R., Michel J. L., and Murphy J. R., 1983. J. Bacteriol, 153:1202-1210.
Rappuoli R. et al, 1990, U.S. Pat. No. 4,925,792.
Metcalf B. J., 1997, U.S. Pat. No. 5,614,382.
Leong D., Coleman K. D., and Murphy J. R., 1983. J Biol Chem, 258:15016-20.
Bishai W. R., Miyanohara A. and Murphy J. R., 1987a. J Bacteriol, 169(4): 1554-1563.
Bishai W. R., Rappuoli R. and Murphy J. R., 1987b. J Bacteriol, 169(11): 5140-5151.
Spilsberg B., Sandvig K., and Walchli S., 2005. Toxicon, 46: 900-906.
Corvaia N., Nguyen T. N. and Beck A., FR 2827606A1 2003.
Dehottay P. M. H. et al, US2008/0193475.
Wolfe H. et al, US2008/0153750.
Mekada E. and Miyamoto S., US 2006/0270600A1.

1-10. (canceled)

11. An isolated nucleic acid molecule which encodes polypeptide CMR197, the nucleotide sequence of which is set forth at SEQ ID NO: 1.

12. The isolated nucleic acid molecule of claim 11, further comprising a nucleotide sequence which encodes a tag polypeptide.

13. The isolated nucleic acid molecule of claim 12, wherein said nucleotide sequence which encodes a tag polypeptide is positioned 5′ of SEQ ID NO: 1.

14. The isolated nucleic acid molecule of claim 12, comprising the nucleotide sequence set forth in SEQ ID NO: 2, SEQ ID NO: 3, or SEQ ID NO: 5.

15. The isolated nucleic acid molecule of claim 12, wherein said tag polypeptide comprises a restriction endonuclease recognition sequence.

16. An expression vector comprising the isolated nucleic acid molecule of claim 11, operably linked to a promoter.

17. Recombinant cell comprising the isolated nucleic acid molecule of claim 11.

18. Recombinant cell comprising the expression vector of claim 16.

19. The recombinant cell of claim 17, wherein said cell is Escherichia coli.

20. The recombinant cell of claim 18, wherein said cell is Escherichia coli.

21. A fusion protein encoded by the isolated nucleic acid molecule of claim 12.

22. A fusion protein encoded by the isolated nucleic acid molecule of claim 14.

23. A method for recombinant production of a CMR197 tag fusion protein, comprising culturing the recombinant cell of claim 20 under conditions favoring production of said CMR197 tag fusion protein, and isolating said fusion protein.

24. A method for recombinant production of CMR197 tag fusion protein, comprising culturing the recombinant cell of claim 20 under condition favoring production of CMR197 tag fusion protein, and claiming said tag protein thereform.

25. Composition comprising the fusion protein of claim 12 and a pharmaceutically acceptable carrier.
